SEQUENCE LISTING (25 sequences)

SEQ ID NO: 1 - protein, 177 aa, Homo sapiens
  SIGNAL 1..25
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - DNA, 22 bp, synthetic construct
  misc_feature 1..22: note = Oligonucleotide
  source 1..22: mol_type = other DNA
cactgttcagggagctcaataa

SEQ ID NO: 3 - DNA, 25 bp, synthetic construct
  misc_feature 1..25: note = Oligonucleotide
  source 1..25: mol_type = other DNA
tggagtagtccttgggattatagtc

SEQ ID NO: 4 - DNA, 25 bp, synthetic construct
  misc_feature 1..25: note = Oligonucleotide
  source 1..25: mol_type = other DNA
cctccttttctttgatgttgtgggc

SEQ ID NO: 5 - DNA, 20 bp, synthetic construct
  misc_feature 1..20: note = Oligonucleotide
  source 1..20: mol_type = other DNA
tgagtgcccttctgaaatcg

SEQ ID NO: 6 - DNA, 20 bp, synthetic construct
  misc_feature 1..20: note = Oligonucleotide
  source 1..20: mol_type = other DNA
tgtcgcctgtacagttttcg

SEQ ID NO: 7 - DNA, 24 bp, synthetic construct
  misc_feature 1..24: note = Oligonucleotide
  source 1..24: mol_type = other DNA
ctggtacagagacccccactgcac

SEQ ID NO: 8 - DNA, 20 bp, synthetic construct
  misc_feature 1..20: note = Oligonucleotide
  source 1..20: mol_type = other DNA
tcatcagtttccagccagtg

SEQ ID NO: 9 - DNA, 28 bp, synthetic construct
  misc_feature 1..28: note = Oligonulceotide
  source 1..28: mol_type = other DNA
gtataaatgtctccatagttgtagctgc

SEQ ID NO: 10 - DNA, 28 bp, synthetic construct
  misc_feature 1..28: note = Oligonucleotide
  source 1..28: mol_type = other DNA
tcctgagagtgagacttcctgtcccatc

SEQ ID NO: 11 - DNA, 18 bp, synthetic construct
  misc_feature 1..18: note = Oligonucleotide
  source 1..18: mol_type = other DNA
agtgcatcgagggactcc

SEQ ID NO: 12 - DNA, 22 bp, synthetic construct
  misc_feature 1..22: note = Oligonucleotide
  source 1..22: mol_type = other DNA
cagtttttacaaccctgctctg

SEQ ID NO: 13 - DNA, 22 bp, synthetic construct
  misc_feature 1..22: note = Oligonucleotide
  source 1..22: mol_type = other DNA
ctcttgacccggcttgcaatcc

SEQ ID NO: 14 - DNA, 18 bp, synthetic construct
  misc_feature 1..18: note = Oligonucleotide
  source 1..18: mol_type = other DNA
gaactggcaaaaggctgg

SEQ ID NO: 15 - DNA, 22 bp, synthetic construct
  misc_feature 1..22: note = Oligonucleotide
  source 1..22: mol_type = other DNA
ccttcaaggcttcaaagagttt

SEQ ID NO: 16 - DNA, 30 bp, synthetic construct
  misc_feature 1..30: note = Oligonucleotide
  source 1..30: mol_type = other DNA
cattgagagccagatcgtctccttctactt

SEQ ID NO: 17 - DNA, 25 bp, synthetic construct
  misc_feature 1..25: note = Oligonucleotide
  source 1..25: mol_type = other DNA
gaccgatcttttaggtatgaaaacg

SEQ ID NO: 18 - DNA, 22 bp, synthetic construct
  misc_feature 1..22: note = Oligonucleotide
  source 1..22: mol_type = other DNA
gtggtttttgaagcttcagcat

SEQ ID NO: 19 - DNA, 21 bp, synthetic construct
  misc_feature 1..21: note = Oligonucleotide
  source 1..21: mol_type = other DNA
acggcgagcctcgagagctag

SEQ ID NO: 20 - DNA, 19 bp, synthetic construct
  misc_feature 1..19: note = Oligonucleotide
  source 1..19: mol_type = other DNA
gtgtcctagtgggtgtccc

SEQ ID NO: 21 - DNA, 17 bp, synthetic construct
  misc_feature 1..17: note = Oligonucleotide
  source 1..17: mol_type = other DNA
gccctccttcagaggct

SEQ ID NO: 22 - DNA, 19 bp, synthetic construct
  misc_feature 1..19: note = Oligonucleotide
  source 1..19: mol_type = other DNA
gctgctggcctgggtccta

SEQ ID NO: 23 - DNA, 18 bp, synthetic construct
  misc_feature 1..18: note = Oligonucleotide
  source 1..18: mol_type = other DNA
ggagtgcgccgagacaac

SEQ ID NO: 24 - DNA, 19 bp, synthetic construct
  misc_feature 1..19: note = Oligonucleotide
  source 1..19: mol_type = other DNA
actacgtccggcgttccat

SEQ ID NO: 25 - DNA, 28 bp, synthetic construct
  misc_feature 1..28: note = Oligonucleotide
  source 1..28: mol_type = other DNA
tggcatgacactacgaccaacacgatct

(19) United States
(12) Patent Application Publication
(10) Pub. No.: US 2024/0398986 A1
(43) Pub. Date: Dec. 5, 2024

(54) An oncolytic virus vector coding for interleukin-7 (IL-7) polypeptide

(71) Applicant: TILT Biotherapeutics Oy, Helsinki (FI)

(72) Inventors: Tatiana Kudling, Helsinki (FI); Akseli Hemminki, Helsinki (FI); James Clubb, Helsinki (FI); Dafne Quixabeira, Helsinki (FI); Riikka Havunen, Helsinki (FI)

(21) Appl. No.: 18/698,071

(22) PCT Filed: Oct. 4, 2022
(86) PCT No.: PCT/FI2022/050662

§ 371 Date: Apr. 3, 2024

(30) Foreign Application Priority Data
Oct. 4, 2021 (FI) 20216026

(51) Int. Cl. A61K 48/00; A61K 38/19; A61K 38/20; A61P 35/00; C12N 15/86

(52) U.S. Cl. CPC A61K 48/005; A61K 38/191; A61K 38/2086; A61P 35/00; C12N 15/86; C12N 2710/10022; C12N 2710/10043

(57) ABSTRACT

The present invention provides an oncolytic adenoviral vector comprising a nucleic acid sequence encoding an interleukin 7 (IL-7) polypeptide or a variant thereof as a transgene. The present invention also provides a pharmaceutical composition comprising said oncolytic vector and at least one of the following: physiologically acceptable carriers, buffers, excipients, adjuvants, additives, antiseptics, preservatives, filling, stabilising and/or thickening agents. A particular aim of the present invention is to provide said oncolytic viral vector or pharmaceutical composition for use in the treatment of cancer or tumor, preferably a solid tumor.

### FIELD OF THE INVENTION

The present invention relates to the fields of life sciences and medicine. Specifically, the invention relates to cancer therapies of humans. More specifically, the present invention relates to an oncolytic viral vector comprising a nucleic acid sequence encoding an interleukin-7 (IL-7 or IL7) polypeptide.

### BACKGROUND OF THE INVENTION

IL-7 is one of the key cytokines involved in immune cell expansion and proliferation. Its main function is to maintain the survival of naive and memory T cells and their diversity. IL-7 can improve the effector functions of T cells via repression of negative regulators of T cells activation and increase IFNγ production (Rosenberg et al. 2006). Conversely, IL-7 antagonizes immunosuppressive pathways via several mechanisms. It prevents the activation of regulatory T cells and inhibits their ability to suppress effector cells (Pellegrini et al. 2012). Further, IL-7 abrogates inhibition of T cells proliferation and prevents their exhaustion (Heninger at al. 2012).

Recombinant IL-7 showed promising results in pre-clinical experiments, but in a phase I clinical trials off-target toxicity caused dose limiting toxicities. Only one out of sixteen patients had tumor response (Sportès et al. 2010).

In the prior art, Huang et al. 2021 discloses an oncolytic adenoviral vector encoding IL-7 where the backbone of the vector is adenovirus serotype 5. This vector was used in combination with adoptive cell therapy (ACT) for the treatment of glioblastoma. The treatment led to prolonged survival of the tumor-bearing mice. However, the authors state that one major limitation of the study is the design of the IL-7 loaded oncolytic adenoviral vector, which hinders it from infecting mouse-derived glioblastoma cells. Indeed, the adenoviral vector used in the study enters cells through CXAR receptor, which is not expressed by all tumor cells.

Nakao et al. 2020 discloses a tumor specific vaccinia virus vector carrying both IL-7 and IL-12 genes. The vector was used in combination with anti-PD-1 and anti-CTLA4 antibodies to treat contralateral tumors in mouse model leading to tumor regression. The authors however state that the checkpoint inhibitors can hinder the replication of vaccinia virus.

After years of development, oncolytic viruses are currently starting to be used as cancer therapeutics. Although there have been discoveries relating to the mechanisms of action and factors that influence the efficacy of the viruses, there is still a need to identify pathways that determine the overall response to virotherapy. In clinical trials, oncolytic viruses have demonstrated a favorable safety profile and promising efficacy.

WO2014170389 relates to oncolytic adenoviral vectors alone or together with therapeutic compositions for therapeutic uses and therapeutic methods for cancer. For instance, a separate administration of adoptive cell therapeutic composition and oncolytic adenoviral vectors is disclosed. Adoptive cell therapies are a potent approach for treating cancer but also for treating other diseases such as infections and graft versus host disease. Adoptive cell transfer is the passive transfer of ex vivo grown cells, most commonly immune-derived cells, into a host with the goal of transferring the immunologic functionality and characteristics of the transplant. WO2014170389 also discloses nucleic acid sequences of oncolytic adenoviral vectors.

WO2016146894 discloses an oncolytic adenoviral vector encoding a bispecific monoclonal antibody.

EP 3858369 relates to a cancer therapy by combination use of an oncolytic vaccinia virus and an immune checkpoint inhibitor. The virus encodes two interleukins, IL-7 and IL-12, and is administered with an immune checkpoint inhibitor to further improve its antitumoral effect.

There is still room for improving oncolytic viral treatments for cancer patients, especially in patients with a significant metastasis burden. Further characterization of pathways related to the activity of oncolytic viruses could reveal potential targets for improving the efficacy of virotherapy. Therefore, the efficacy of oncolytic viral vectors, either alone or together with other therapies, can still be improved. The present invention provides efficient tools and methods for cancer therapeutics by utilizing specific viral vectors, with e.g. adoptive cell therapies and/or immune checkpoint inhibitors.

### SUMMARY OF THE INVENTION

The aim of this invention is to overcome the limitations seen in the use of oncolytic adenoviruses in cancer treatments. Accordingly, an object of the present invention is to provide simple methods and tools for overcoming the problems of inefficient, unsafe and unpredictable cancer therapies. In embodiments of the invention, novel approaches and means for cancer therapy are thus provided. The objects of the invention are achieved by specific viral vectors, methods and arrangements, which are characterized by what is stated in the independent claims. The specific embodiments of the invention are disclosed in the dependent claims.

Specifically, the present invention provides an oncolytic adenoviral vector comprising a nucleic acid sequence encoding an interleukin 7 (IL-7) polypeptide as a transgene. The present invention also provides a pharmaceutical composition comprising said oncolytic vector and at least one of the following: physiologically acceptable carriers, buffers, excipients, adjuvants, additives, antiseptics, preservatives, filling, stabilising and/or thickening agents. A particular aim of the present invention is to provide said oncolytic viral vector or pharmaceutical composition for use in the treatment of cancer or tumor, preferably a solid tumor.

### BRIEF DESCRIPTION OF THE DRAWINGS

FIG. 1. Ad5/3-E2F-d24-IL7 functionality in vitro. (A) A schematic presentation of chimeric 5/3 oncolytic adenovirus containing an E2F promoter; 24-base-pair deletion in E1A; human IL7 transgene inserted in the E3 region; and an Ad3 serotype knob in the Ad5 fiber. (B) Relative human cancer cell viability after addition of 1, 10, 100 or 1000 virus particles (VP)/cell at day 4 after infection in epithelial adenocarcinoma (A549) and rhabdomyosarcoma (RD). No major differences between Ad5/3-E2F-d24-IL7 and Ad5/3-E2F-d24 tumor cell killing ability were observed, thus indicating that the presence of the IL7 transgene does not reduce the oncolytic potency of Ad5/3-E2F-d24-IL7 in human cancer cells. Statistical significance compared to mock is represented as *p<0.05, **p<0.01, ***p<0.001, and ****p<0.0001. (C) Relative hamster cancer cell viability after addition of 100, 1000, 5000 or 10000 VP/cell at day 5 after infection in hamster lung cancer (HT100) and hamster leiomyosarcoma (DDT1-MF2) or, at day 8 after infection in hamster pancreatic cancer (HapT1). There were no major differences between Ad5/3-E2F-d24-IL7 and Ad5/3-E2F-d24 cell killing ability, thus indicating that presence of IL7 transgene does not reduce the oncolytic potency of Ad5/3-E2F-d24-IL7 in hamster cancer cells. Statistical significance compared to mock is represented as **p<0.01, ***p<0.001, and ****p<0.0001. (D) IL7 expression was analysed from the transfected cancer cells. IL7 concentration was measured in cell supernatants harvested on day 3 after 1000 VP/cell infection (A549 and RD) or 10000 VP/cell infection (HT100 and DDT1-MF2). The analysis showed that Ad5/3-E2F-d24-hIL7 is able to induce the expression of IL7 in multiple cancer cell lines. (E) IL7 bioactivity measured after 1000 VP/cell infection of A549 cell line. The supernatant was filtered, diluted with growth media and applied on IL7-dependent murine cell line 2E8. Recombinant murine IL7 (rmIL7) and murine IL7 (rhIL7) were used as controls in 20 ng/ml. These data indicates that the IL-7 produced by cancer cells upon infection with Ad5/3-E2F-d24-IL7 is functional. In vitro data sets performed in triplicates and all data are shown as means±SEM.

FIG. 2. Ad5/3-E2F-d24-IL7 efficacy in vivo. (A) Tumor growth until day 30 from experimental groups treated with PBS, Ad5/3-E2F-d24 or Ad5/3-E2F-d24-IL7 virus. Tumor volumes were normalized against day 0. Data is presented as median+range. Statistical significance from normalized tumor volumes is represented as *p<0.05, **p<0.01, ***p<0.001, and ****p<0.0001. Treatment with Ad5/3-E2F-d24-IL7 caused a significant reduction of tumor volume relative to experimental controls (unarmed virus (Ad5/3-D24-E2F) and mock), thus providing the best antitumor efficacy. (B) Immune-related gene expression changes in tumor digests. Total RNA was isolated from tumor tissue with subsequent cDNA synthesis and quantitative real-time PCR. PCRs were carried out in duplicates, and data was normalized against mock. Data is presented as mean+SEM. Tumors receiving Ad5/3-E2F-d24-IL7 treatment demonstrated increased transcription of immune-related genes relative to mock-treated animals, indicating that the IL-7 encoding virus is capable of inducing superior local immune activation.

FIG. 3. Lytic capability and replication of IL7 armed adenovirus in cancer patient ex vivo tumor cultures (A) Viability of tumor digests from ovarian (HUSOV4 and OvCaS) and head and neck (HUSHN11) cancer patients was assessed at different days after 100 VP/cell oncolytic virus infection. Cell viability data is normalized against the uninfected mock. Experiments were performed in triplicates. Statistical significance is represented as *p<0.05, **p<0.01, ***p<0.001, and ****p<0.0001. No major differences between Ad5/3-E2F-d24-IL7 and Ad5/3-E2F-d24 tumor cell killing ability were observed in most cancer patient samples, indicating that the presence of the IL7 transgene does not reduce the oncolytic potency of Ad5/3-E2F-d24-IL7 in most cancer patients' tumor cultures. (B) Ad5/3 replication evaluation through quantitative real-time PCR from ovarian (HUSOV4 and OvCaS) and head and neck (HUSHN11 an HUSHN10) samples. Viral copy number was normalized against the amount of genomic DNA in the sample, determined by the expression level of human β-actin. PCRs were carried out in duplicates. The data shows similar patterns of virus DNA levels over time between Ad5/3-E2F-d24 and Ad5/3-E2F-d24-IL7, indicating that the IL-7 transgene does not hinder virus replication. (C) IL7 protein concentration in supernatants from ovarian (HUSOV4 and OvCaS) and head and neck (HUSHN15 an HUSHN17) samples, measured by Cytokine Bead Array (CBA) assay. Experiment was carried out in triplicates. This indicates that IL-7 is produced in human cancer patients' samples infected with Ad5/3-E2F-d24-IL7. All data is presented as mean+SEM.

FIG. 4. Evaluation of cytokines and chemokines in tumor microenvironment. (A) Level of pro-inflammatory cytokines, (C) anti-inflammatory cytokines and (E) chemokines obtained from ovarian cancer samples HUSOV4, HUSOV5 and OvCaS, after 3 days infection with MOI 100 of oncolytic adenoviruses. Pooled (B) pro-inflammatory and (D) anti-inflammatory changes and (F) overall ratio of pro-to anti-inflammatory cytokines. The increased content in pro-inflammatory cytokines and chemokines shows the ability of the IL-7 virus to better polarize the microenvironment of human cancer patients' samples towards immune stimulation, relative to controls. In addition, a general minimal impact or reduction in anti-inflammatory cytokine content was observed in IL-7 virus-treated wells. All data was normalized against mock. All experiments were performed in triplicates, and resulting data is presented as mean+SEM. Statistical significance is represented as *p<0.05, **p<0.01, ***p<0.001, and ****p<0.0001.

FIG. 5. Evaluation of infiltrating CD4+ and CD8+ T-cell activation and cytotoxicity in ovarian cancer ex vivo samples (HUSOV4 and OvCaS). (A) Frequency of CD69+cells in CD4+ and CD8+ cells populations. (B) Expression level (designated as MFI, mean fluorescence intensity) of activation receptor CD69 on CD4+ and CD8+ cells. (C) Frequency of perforin and granzyme B expressing CD4+ and CD8+ cells in HUSOV4 sample. (D) Frequency of perforin and granzyme B expressing CD4+ and CD8+ cells in OvCaS sample. Overall, the data suggests that treatment of patient-derived ovarian ex vivo tumor cultures with Ad5/3-E2F-d24-hIL7 activates and increases the frequency of T-cell subpopulations. All flow cytometry experiments were run in duplicates, and resulting data is presented as mean+SEM. Statistical significance is represented as *p<0.05, **p<0.01.

FIG. 6. Relative cancer cell viability of patient-derived renal cell carcinoma (RCC) samples (HUSRenca5) treated with Ad5/3-E2F-d24-IL7 (TILT-517) and immune-checkpoint inhibitors (anti-PD1 and anti-PD-L1). (A) Overall cell viability of HUSRenca5 tumor cells at Day 1, 2, 3, 4, and 5. (B) Detailed point of view of tumor cell killing on Day 2 and (C) Day 5 accessed by MTS assay. In (B) and (C), the samples are from left to right: Mock, TILT-517, antiPD-1, antiPD-L1, TILT-517 +antiPD-1, TILT-517 +antiPD-L1; *p<0.05, **p<0.01 and ***p<0.001.

### DETAILED DESCRIPTION OF THE INVENTION

### Interleukin 7 (IL-7) and Variants Thereof

As used herein, “IL-7” or “IL7” means wild-type IL-7 isoform 1 polypeptide, whether native or recombinant, or a nucleic acid (i.e. a gene) encoding said polypeptide. Mature human IL-7 occurs as a 152 amino acid sequence (without the signal peptide, consisting of an additional 25 N-terminal amino acids). The amino acid sequence of human IL-7 (SEQ ID NO: 1) is found in Genbank under accession number NP_000871.1. In its broadest sense, terms “IL-7” or “IL7” may also refer to any IL-7 variant which is suitable for cancer therapy.

As used herein, “IL-7 variant”, “variant IL-7”, “vIL7” or “vIL-7” means a polypeptide or a nucleic acid (i.e. a gene) encoding said polypeptide, wherein specific variations or modifications such as substitutions to the interleukin-7 polypeptide have been made or found. The term “polypeptide” refers herein to any chain of amino acid residues, regardless of its length or post-translational modification (e.g., glycosylation or phosphorylation). The variant IL-7 polypeptides can also be characterized by amino acid insertions, deletions, substitutions and modifications at one or more sites in or at the other residues of the native IL-7 polypeptide chain. Any such insertions, deletions, substitutions and modifications may result in a variant IL-7 that preferably exhibits modified binding to receptor subunit IL-7R or its components with the intent of improving properties of the IL-7 variant for cancer therapy. Exemplary variants can include substitutions of 1, 2, 3, 4, 5, 6, 7, 8, 9, 10 or more amino acids. Variants may also include conservative modifications and substitutions at other positions of IL-7 (i.e., those that have a minimal effect on the activity or secondary or tertiary structure of the variant). The IL-7 variants may also include natural isoforms (Vudattu et al. 2008). The sequences for natural IL-7 isoform precursors can be found in Genbank under accession numbers NP_001186815.1 (isoform 2), NP_001186816.1 (isoform 3) and NP_001186817.1 (isoform 4), while isoform 2 has a 44-amino acid deletion in positions 77-120, isoform 3 has 18-amino acid deletion in positions 121-138 and isoform 4-62-amino acid deletion in position 76-138 compared to isoform 1 sequence.

An exemplary variant IL-7 polypeptide includes an amino acid sequence that is at least about 80% identical to SEQ ID NO: 1 which binds the IL-7R with modified affinity that is higher or lower than the affinity with which the polypeptide represented by SEQ ID NO: 1 binds the IL-7R. Exemplary variant IL-7 polypeptides can be at least about 50%, at least about 65%, at least about 70%, at least about 80%, at least about 85%, at least about 87%, at least about 90%, at least about 95%, at least about 97%, at least about 98%, or at least about 99% identical to wild-type IL-7. The variant polypeptide can comprise a change in the number or content of amino acid residues. For example, the variant IL-7 can have a greater or a lesser number of amino acid residues than wild-type IL-7. Alternatively, or in addition, an exemplary variant polypeptide can contain a substitution of one or more amino acid residues that are present in the wild-type IL-7.

In another embodiment, IL-7 polypeptides can also be prepared as fusion or chimeric polypeptides that include an IL-7 polypeptide and another heterologous polypeptide. A chimeric polypeptide including an IL-7 and an antibody or antigen-binding portion thereof can be generated. The antibody or antigen-binding component of the chimeric protein can serve as a targeting moiety. For example, it can be used to localize the chimeric protein to a particular subset of cells or target molecule.

The present invention is particularly directed to a design of an oncolytic viral vector comprising nucleic acid sequence encoding any of the above-mentioned IL-7 polypeptides as a transgene.

### Viral Vectors

Oncolytic viral vectors are therapeutically useful anticancer viruses that can selectively infect, replicate, and destroy cancer cells. Most current oncolytic viruses are adapted or engineered for tumour selectivity, although there are viruses, such as reovirus and Mumps virus, having natural preference for cancer cells. Many engineered oncolytic viral vectors take advantage of tumor-specific promoter elements making them replication competent only in cancer cells. Surface markers expressed selectively by cancer cells can also be targeted by using them as receptors for virus entry. A number of viruses including adenovirus, reovirus, measles, herpes simplex virus, Newcastle disease virus and vaccinia virus have now been clinically tested as oncolytic agents.

Preferably, the oncolytic vector used in the present invention is an adenoviral vector suitable for treating a human or animal. As used herein “an oncolytic adenoviral vector” refers to an adenoviral vector capable of infecting and killing cancer cells by selective replication in tumor versus normal cells. As used herein, the expression “adenovirus serotype 5 (Ad5) nucleic acid backbone” refers to the genome of Ad5. Similarly, “adenovirus serotype 3 (Ad3) nucleic acid backbone” refers to the genome of Ad3. “Ad5/3 vector” refers to a chimeric vector comprising or having parts of both Ad5 and Ad3 vectors.

In one embodiment of the invention, the adenoviral vectors are vectors of human viruses. Specifically, the adenoviral vector is an Ad5/3 vector. In one embodiment the backbone is Ad5 nucleic acid backbone further comprising an Ad3 fiber knob. In other words, the construct has the fiber knob from Ad3 while the remainder or the most of the remainder of the genome is from Ad5 (see, e.g., WO2014170389).

The adenoviral vectors may be modified in any way known in the art, e.g. by deleting, inserting, mutating or modifying any viral regions. The vectors are made tumor specific with regard to replication. For example, the adenoviral vector may comprise modifications in E1, E3 and/or E4 such as insertion of tumor specific promoters (e.g. to drive E1), deletions of areas (e.g. the constant region 2 of E1 as used in “Δ24”, E3/gp19k, E3/6.7k) and insertion of a transgene or transgenes.

In a specific embodiment, the E1B 19K gene, generally known to support replication of adenoviral vectors, has a disabling deletion dE1B 19K in the present vectors as described in WO2020249873.

One approach for generation of a tumor specific oncolytic adenovirus is engineering a 24 base pair (bp) deletion (“Δ24” or “d24”) affecting the constant region 2 (CR2) of E1. In wild type adenovirus CR2 is responsible for binding the cellular Rb tumor suppressor/cell cycle regulator protein for induction of the synthesis(S) phase i.e. DNA synthesis or replication phase. The interaction between pRb and E1A requires amino acids 121 to 127 of the E1A protein conserved region. The vector may comprise a deletion of nucleotides corresponding to amino acids 122-129 of the vector according to Heise C. et al. (2000, Nature Med 6, 1134-1139) and Fueyo J. et al. (2000, Oncogene 19 (1): 2-12). Viruses with the Δ24 are known to have a reduced ability to overcome the G1-S checkpoint and replicate efficiently only in cells where this interaction is not necessary, e.g. in tumor cells defective in the Rb-p16 pathway, which includes most, if not all, human tumors. In one embodiment of the invention the vector comprises a 24 bp deletion (“Δ24” or “d24”) in the Rb binding constant region 2 of adenoviral E1.

It is also possible to replace E1A endogenous viral promoter for example by a tumor specific promoter. For instance, E2F1 (e.g. in Ad5 based vector) or hTERT (e.g. in Ad3 based vector) promoter can be utilized in the place of E1A endogenous viral promoter. The vector may comprise E2F1 promoter for tumor specific expression of E1A. The E1A promoter can also be deleted.

The E3 region is nonessential for viral replication ex vivo, but the E3 proteins have an important role in the regulation of host immune response i.e. in the inhibition of both innate and specific immune responses. In one embodiment of the invention the deletion of a nucleic acid sequence in the E3 region of the oncolytic adenoviral vector is a deletion of viral gp19k and 6.7k reading frames. The gp19k/6.7K deletion in E3 refers to a deletion of 965 base pairs from the adenoviral E3A region. In a resulting adenoviral construct, both gp19k and 6.7K genes are deleted (Kanerva A et al. 2005, Gene Therapy 12, 87-94). The gp19k gene product is known to bind and sequester major histocompatibility complex I (MHC1, known as HLA1 in humans) molecules in the endoplasmic reticulum, and to prevent the recognition of infected cells by cytotoxic T-lymphocytes. Since many tumors are deficient in HLA1/MHC1, deletion of gp19k increases tumor selectivity of viruses (virus is cleared faster than wild type virus from normal cells but there is no difference in tumor cells). 6.7K proteins are expressed on cellular surfaces and they take part in downregulating TNF-related apoptosis inducing ligand (TRAIL) receptor 2.

In one embodiment of the invention, the transgene, i.e. a gene encoding interleukin 7 (IL7), is placed into a gp19k/6.7k deleted E3 region, under the E3 promoter. This restricts transgene expression to tumor cells that allow replication of the virus and subsequent activation of the E3 promoter. In a specific embodiment a nucleic acid sequence encoding interleukin 7 is inserted into the place of the deleted nucleic acid sequence of viral gp19k and 6.7k reading frames. In another embodiment of the invention E3 gp19k/6.7k is kept in the vector but one or many other E3 areas have been deleted (e.g. E3 9-kDa, E3 10.2 kDa, E3 15.2 kDa and/or E3 15.3 kDa).

E3 promoter may be any exogenous (e.g. CMV or E2F promoter) or endogenous promoter known in the art, specifically the endogenous E3 promoter. Although the E3 promoter is chiefly activated by replication, some expression occurs when E1 is expressed. As the selectivity of 424 type viruses occurs post E1 expression (when E1 is unable to bind Rb), these viruses do express E1 also in transduced normal cells. Thus, it is of critical importance to regulate also E1 expression to restrict E3 promoter mediated transgene expression to tumor cells.

Specific embodiments of the invention include oncolytic adenoviral vectors (e.g. Ad5/Ad3 vectors) whose replication is restricted to the Retinoblastoma (Rb)/p16 pathway by dual selectivity devices: an E2F (e.g. E2F1) tumor specific promoter placed in front of the adenoviral E1A gene which has been mutated in constant region 2, so that the resulting E1A protein is unable to bind Rb in cells. Furthermore, the fiber is modified by 5/3 chimerism to allow efficient entry into tumor cells.

In a specific embodiment of the invention the oncolytic adenoviral vector comprises:
1) a 24 bp deletion (Δ24) in the Rb binding constant region 2 of adenoviral E1
2) a nucleic acid sequence deletion of viral gp19k and 6.7k reading frames; and
3) a nucleic acid sequence encoding an interleukin 7 (IL7) transgene in the place of the deleted nucleic acid sequence as defined in point 2).

In the Experimental Section below, we constructed and characterized an oncolytic adenovirus based on Ad5/3-E2F-d24 backbone and armed it with IL7. The virus has an E2F promoter and a 24-base pair deletion in the E1A constant region 2 (“D24”) to enable its replication only in rb/p16 pathway-defective cells, which is one of the common features for all cancer cells. E1B region is deleted to induce cancer cell apoptosis (dE1B 19K). Moreover, to improve its ability to transduce cancer cells and enhance its antitumor efficacy, the virus features fiber knob from serotype 3, while the rest of the genome derives from serotype 5. Most importantly, Ad5/3 viruses have good safety profile in humans. Preferably, oncolytic virus armed with IL-7 is used with concomitant T-cell therapy or checkpoint inhibitors, as a potential platform to safely and effectively treat currently incurable solid tumors. In particular, tumor types where T-cells are dysfunctional are preferably treated.

In an embodiment, the present invention is directed to an oncolytic viral vector, preferably an oncolytic adenoviral vector, comprising a nucleic acid sequence encoding an interleukin 7 (IL7) transgene.

In a preferred embodiment, the backbone of the oncolytic adenoviral vector is an adenovirus serotype 5 (Ad5) or serotype 3 (Ad3) nucleic acid backbone.

In a more preferred embodiment, said nucleic acid sequence encoding an interleukin 7 (IL7) transgene is in the place of a deleted nucleic acid sequence in the E3 region of said oncolytic adenoviral vector. Most preferably, the deletion of a nucleic acid sequence in the E3 region is a deletion of viral gp19 k and 6.7 k reading frames.

In another preferred embodiment, the vector also comprises a 24 bp deletion (Δ24) in the adenoviral E1 sequence of said oncolytic adenoviral vector.

In another preferred embodiment, the vector also comprises a disabling deletion of E1B (dE1B 19K).

In another preferred embodiment, the vector also comprises an Ad5/3 fiber knob.

In another preferred embodiment, the vector comprises nucleic acid sequence encoding a further transgene. More preferably, the further transgene is encoding a cytokine. In an embodiment, the cytokine is selected from the list consisting of: TNFalpha, interferon alpha, interferon beta, interferon gamma, complement C5a, CD40L, IL-2, IL-12, IL-23, IL-21, IL-15, IL-17, IL-18, CCL1, CCL11, CCL12, CCL13, CCL14-1, CCL14-2, CCL14-3, CCL15-1, CCL15-2, CCL16, CCL17, CCL18, CCL19, CCL2, CCL20, CCL21, CCL22, CCL23-1, CCL23-2, CCL24, CCL25 -1, CCL25-2, CCL26, CCL27, CCL28, CCL3, CCL3L1, CCL4, CCL4L1, CCL5 (=RANTES), CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCR1, CXCR2, CXCR4, CXCR5, CXCR6, CXCR7 and XCL2.

In a more preferred embodiment, the cytokine is TNFalpha or IL-15.

The viral vectors utilized in the present inventions may also comprise other modifications than described above. Any additional components or modifications may optionally be used but are not obligatory for the present invention.

Insertion of exogenous elements may enhance effects of vectors in target cells. The use of exogenous tissue or tumor-specific promoters is common in recombinant vectors and they can also be utilized in the present invention.

### Adoptive Cell Therapy

One approach of the present invention is the development of a treatment for patients with cancer using the transfer of immune effector cells that are capable of reacting with and destroying the cancer. Isolated immune effector cells such as tumor-infiltrating lymphocytes (TILs) are grown in culture to large numbers and infused into the patient. In the present invention oncolytic vectors encoding an interleukin 7 (IL7) transgene may be utilized for increasing the effect of immune effector cells. As used herein “increasing the efficacy of adoptive cell therapy” refers to a situation, wherein the oncolytic vector of the invention is able to cause a stronger therapeutic effect in a subject when used together with an adoptive cell therapeutic composition compared to the therapeutic effect of the adoptive cell therapeutic composition alone. A specific embodiment of the invention is a method of treating cancer in a subject, wherein the method comprises administration of an oncolytic vector of the invention to a subject, said method further comprising administration of adoptive cell therapeutic composition to the subject. Adoptive cell therapeutic composition and the vectors of the invention are administered separately. Separate administrations of an adoptive cell therapeutic composition and adenoviral vectors may be preceded by myeloablating or non-myeloablating preconditioning chemotherapy and/or radiation. The adoptive cell therapy treatment is intended to reduce or eliminate cancer in the patient.

A specific embodiment of the invention relates to therapies with adenoviral vectors and an adoptive cell therapeutic composition, e.g. tumor-infiltrating lymphocytes (TIL), T-cell receptor (TCR)-modified lymphocytes or chimeric antigen receptor (CAR) modified lymphocytes. T-cell therapies in particular, but also any other adoptive cell therapies, such as natural killer (NK) or CAR-NK cell therapies, may be utilized in the present invention. Indeed, according to the present invention the adoptive cell therapeutic composition may comprise unmodified cells, such as in TIL therapy, or genetically modified cells. There are two common ways to achieve genetic targeting of T-cells to tumor-specific targets. One is transfer of a T-cell receptor (TCR) with known antigen specificity and with matched human leukocyte antigen (HLA, known as major histocompatibility complex in rodents) type. The other is modification of cells with artificial receptor such as chimeric antigen receptors (CAR). This approach is not dependent on HLA and is more flexible with regard to targeting molecules. For example, single chain antibodies can be used and CARs can also incorporate costimulatory domains. However, the targets of CAR cells need to be on the membrane of target cells, while TCR modifications can utilize intracellular targets.

As used herein “adoptive cell therapeutic composition” refers to any composition comprising cells suitable for adoptive cell transfer. In one embodiment of the invention the adoptive cell therapeutic composition comprises a cell type selected from a group consisting of a TIL, TCR (i.e. heterologous T-cell receptor) modified lymphocytes and CAR (i.e. chimeric antigen receptor) modified lymphocytes. In another embodiment of the invention, the adoptive cell therapeutic composition comprises a cell type selected from a group consisting of T-cells, CD8+ cells, CD4+ cells, NK-cells, dendritic cells, gamma-delta T-cells, regulatory T-cells and peripheral blood mononuclear cells. In another embodiment, TILs, T-cells, CD8+ cells, CD4+ cells, NK-cells, gamma-delta T-cells, regulatory T-cells or peripheral blood mononuclear cells form the adoptive cell therapeutic composition. In one specific embodiment of the invention the adoptive cell therapeutic composition comprises T cells. As used herein “tumor-infiltrating lymphocytes” or TILs refer to white blood cells that have left the bloodstream and migrated into a tumor. Lymphocytes can be divided into three groups including B cells, T cells and NK cells. In another specific embodiment of the invention the adoptive cell therapeutic composition comprises T-cells which have been modified with target-specific CARs or specifically selected TCRs. As used herein “T-cells” refers to CD3+ cells, including CD4+ helper cells, CD4+ cytotoxic cells, CD8+ cytotoxic T-cells, gamma-delta T cells and NK T cells.

In addition to suitable cells, adoptive cell therapeutic composition used in the present invention may comprise any other agents such as pharmaceutically acceptable carriers, buffers, excipients, adjuvants, additives, antiseptics, filling, stabilising and/or thickening agents, and/or any components normally found in corresponding products. Selection of suitable ingredients and appropriate manufacturing methods for formulating the compositions belongs to general knowledge of a person skilled in the art.

The adoptive cell therapeutic composition may be in any form, such as solid, semisolid or liquid form, suitable for administration. A formulation can be selected from a group consisting of, but not limited to, solutions, emulsions, suspensions, tablets, pellets and capsules. The compositions are not limited to a certain formulation; instead the composition can be formulated into any known pharmaceutically acceptable formulation. The pharmaceutical compositions may be produced by any conventional processes known in the art.

A combination of an oncolytic adenoviral vector of the invention and an adoptive cell therapeutic composition refers to use of an oncolytic adenoviral vector and an adoptive cell therapeutic composition together but as separate compositions. It is clear to a person skilled in the art that an oncolytic adenoviral vector of the present invention and an adoptive cell therapeutic composition are not used as one composition.

Indeed, adenoviral vectors are not used for directly modifying the adoptive cells but for modifying the target tumor, so that the tumor is more amenable to the desired effects of the cellular transplant. In particular, the present invention enhances recruitment of the adoptive transplant to the tumor, and increases its activity there. In a specific embodiment of the invention oncolytic adenoviral vectors and an adoptive cell therapeutic composition of a combination are for simultaneous or sequential, in any order, administration to a subject.

### Immune Checkpoint Inhibitor

Immune checkpoint proteins interact with specific ligands which send a signal into T cells that inhibits T-cell function. Cancer cells exploit this by driving high-level expression of checkpoint proteins on their surface, thereby suppressing the anti-cancer immune response.

An immune checkpoint inhibitor (also referred to as a CPI or ICI) as described herein is any compound capable of inhibiting the function of an immune checkpoint protein. Inhibition includes reduction of function as well as full blockade. In particular, the immune checkpoint protein is a human checkpoint protein. Thus, the immune checkpoint inhibitor is preferably an inhibitor of a human immune checkpoint.

Immune checkpoint proteins include, without limitation, CTLA-4, PD-1 (and its ligands PD-L1 and PD-L2), B7-H3, B7-H4, HVEM, TIM3, GAL9, LAG3, VISTA, KIR, BTLA, TIGIT and/or IDO. The pathways involving LAG3, BTLA, B7-H3, B7-H4, TIM3 and KIR are recognized in the art to constitute immune checkpoint pathways similar to the CTLA-4 and PD-1 dependent pathways. The immune checkpoint inhibitor can be an inhibitor of CTLA-4, PD-1 (and its ligands PD-L1 and PD-L2), B7-H3, B7-H4, HVEM, TIM3, GAL9, LAG3, VISTA, KIR, BTLA, TIGIT and/or IDO. In some embodiments, the immune checkpoint inhibitor is an inhibitor of PD-L1 or PD-1. Preferably, the immune checkpoint inhibitor is a monoclonal antibody that selectively binds to PD-L1, more preferably selected from the group consisting of: BMS-936559, LY3300054, atezolizumab, durvalumab, envafolimab, cosibelimab and avelumab, or a monoclonal antibody that selectively binds to PD-1, more preferably selected from the group consisting of: pembrolizumab, nivolumab, cemiplimab, Sintilimab, Tislelizumab, Spartalizumab, Toripalimab, Dostarlimab, INCMGA00012, AMP-514.

In some embodiments, the immune checkpoint inhibitor of the combination is an antibody. The term “antibody” as used herein encompasses naturally occurring and engineered antibodies as well as full length antibodies or functional fragments or analogs thereof that are capable of binding e.g. the target immune checkpoint or epitope (e.g. retaining the antigen-binding portion). The antibody for use according to the methods described herein may be from any origin including, without limitation, human, humanized, animal or chimeric and may be of any isotype with a preference for an lgG1 or lgG4 isotype and further may be glycosylated or non-glycosylated. The term antibody also includes bispecific or multispecific antibodies so long as the antibody(s) exhibit the binding specificity herein described.

### Cancer

The recombinant vectors of the present invention are replication competent in tumor cells. In one embodiment of the invention the vectors are replication competent in cells, which have defects in the Rb-pathway, specifically Rb-p16 pathway. These defective cells include all tumor cells in animals and humans. As used herein “defects in the Rb-pathway” refers to mutations and/or epigenetic changes in any genes or proteins of the pathway. Due to these defects, tumor cells overexpress E2F and thus, binding of Rb by E1A CR2, that is normally needed for effective replication, is unnecessary. Further selectivity is mediated by the E2F promoter, which only activates in the presence of free E2F, as seen in Rb/p16 pathway defective cells. In the absence of free E2F, no transcription of E1A occurs and the virus does not replicate. Inclusion of the E2F promoter is important to prevent expression of E1A in normal tissues, which can cause toxicity both directly and indirectly through allowing transgene expression from the E3 promoter.

The present invention relates to approaches for treating cancer in a subject. In one embodiment of the invention, the subject is a human or a mammal, specifically a mammal or human patient, more specifically a human or a mammal suffering from cancer.

The approach can be used to treat any cancers or tumors, including both malignant and benign tumors, both primary tumors and metastases may be targets of the approach. In one embodiment of the invention the cancer features tumor-infiltrating lymphocytes. The tools of the present invention are particularly appealing for treatment of metastatic solid tumors featuring tumor-infiltrating lymphocytes. In another embodiment the T-cell graft has been modified by a tumor or tissue specific T-cell receptor or chimeric antigen receptor.

As used herein, the term “treatment” or “treating” refers to administration of at least oncolytic adenoviral vectors to a subject, preferably a mammal or human subject, for purposes which include not only complete cure but also prophylaxis, amelioration, or alleviation of disorders or symptoms related to a cancer or tumor. Therapeutic effect may be assessed by monitoring the symptoms of a patient, tumor markers in blood, or for example a size of a tumor or the length of survival of the patient

In another embodiment of the invention the cancer or tumor is selected from a group consisting of nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, esophagus cancer, gall bladder cancer, head and neck cancer, eye cancer, kidney cancer, Wilms' tumor, Kaposi's sarcoma, prostate cancer, testicular cancer, Hodgkin's disease, non-Hodgkin's lymphoma, skin cancer, mesothelioma, multiple myeloma, ovarian cancer, endocrine pancreatic cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer. Preferably, the cancer or tumor treated is selected from the group consisting of renal cancer, ovarian cancer, bladder cancer, prostate cancer, breast cancer, lung cancer (such as small-cell lung carcinoma, non-small-cell lung carcinoma and squamous non-small-cell lung carcinoma), gastric cancer, soft tissue sarcoma, classical Hodgkin lymphoma, mesothelioma, and liver cancer. In a preferred embodiment of the invention, the cancer or tumor treated is a mesothelin negative cancer or tumor.

Before classifying a human or animal patient as suitable for the therapy of the present invention, the clinician may examine a patient. Based on the results deviating from the normal and revealing a tumor or cancer, the clinician may suggest treatment of the present invention for a patient.

### Pharmaceutical Composition

A pharmaceutical composition of the invention comprises at least one type of viral vector of the invention. Preferably, the present invention provides a pharmaceutical composition containing (a) an oncolytic virus as such or in combination with (b) adoptive cell composition and/or (c) an immune checkpoint inhibitor. The present invention also provides said pharmaceutical combination for use in the treatment of cancer. Furthermore, the composition may comprise at least two, three or four different vectors. In addition to the vector and adoptive cell composition or immune checkpoint inhibitor, a pharmaceutical composition may also comprise other therapeutically effective agents, any other agents such as pharmaceutically acceptable carriers, buffers, excipients, adjuvants, additives, preservatives, antiseptics, filling, stabilising and/or thickening agents, and/or any components normally found in corresponding products. Selection of suitable ingredients and appropriate manufacturing methods for formulating the compositions belongs to general knowledge of a person skilled in the art.

The pharmaceutical composition may be in any form, such as solid, semisolid or liquid form, suitable for administration. A formulation can be selected from a group consisting of, but not limited to, solutions, emulsions, suspensions, tablets, pellets and capsules. The compositions of the current invention are not limited to a certain formulation, instead the composition can be formulated into any known pharmaceutically acceptable formulation. The pharmaceutical compositions may be produced by any conventional processes known in the art.

A pharmaceutical kit of the present invention comprises an oncolytic adenoviral vector encoding an IL-7 as a transgene and one or more immune checkpoint inhibitors. The oncolytic adenoviral vector encoding an IL-7 as a transgene is formulated in a first formulation and said one or more immune checkpoint inhibitors are formulated in a second formulation. Alternatively, the pharmaceutical kit of the present invention comprises an oncolytic adenoviral vector encoding an IL-7 as a transgene in the first formulation and an adoptive cell composition in the second formulation. In another embodiment of the invention the first and the second formulations are for simultaneous or sequential, in any order, administration to a subject. In another embodiment, said kit is for use in the treatment of cancer or tumor.

### Administration

The vector or pharmaceutical composition of the invention may be administered to any mammal subject. In a specific embodiment of the invention, the subject is a human. A mammal may be selected from a group consisting of pets, domestic animals and production animals.

Any conventional method may be used for administration of the vector or composition to a subject. The route of administration depends on the formulation or form of the composition, the disease, location of tumors, the patient, comorbidities and other factors. Accordingly, the dose amount and dosing frequency of each therapeutic agent in the combination depends in part on the particular therapeutic agent, the severity of the cancer being treated, and patient characteristics. Preferably, a dosage regimen maximizes the amount of each therapeutic agent delivered to the patient consistent with an acceptable level of side effects.

The effective dose of vectors depends on at least the subject in need of the treatment, tumor type and location of the tumor and stage of the tumor. The dose may vary for example from about 1×10⁸ viral particles (VP) to about 1×10¹⁴ VP, specifically from about 5×10⁹ VP to about 1×10¹³ VP and more specifically from about 3×10⁹ VP to about 2×10¹² VP. In one embodiment oncolytic adenoviral vectors coding for an IL-7 are administered in an amount of 1×10¹⁰-1×10¹⁴ virus particles. In another embodiment of the invention the dose is in the range of about 5×10¹⁰-5×10¹¹ VP.

In one embodiment of the invention, the administration of oncolytic virus is conducted through an intratumoral, intra-arterial, intravenous, intrapleural, intravesicular, intracavitary, intranodal or intraperitoneal injection, or an oral administration. Any combination of administrations is also possible. The approach can give systemic efficacy despite local injection.

In one embodiment of the invention, the separate administration(s) of (a) an oncolytic adenoviral vector encoding an IL-7 as a transgene and (b) one or more immune checkpoint inhibitors to a subject is (are) conducted simultaneously or consecutively, in any order. This means that (a) and (b) may be provided in a single unit dosage form for being taken together or as separate entities (e.g. in separate containers) to be administered simultaneously or with a certain time difference. This time difference may be between 1 hour and 2 weeks, preferably between 12 hours and 3 days, more preferably up to 24 or 48 hours. In a preferred embodiment, the first administration of the adenoviral vector is conducted before the first administration of the immune checkpoint inhibitor. In addition, it is possible to administer the virus via another administration way than the immune checkpoint inhibitor. In this regard, it may be advantageous to administer either the virus or immune checkpoint inhibitor intratumorally and the other systemically or orally. In a particular preferred embodiment, the virus is administered intratumorally and the immune checkpoint inhibitor intravenously. In another particular preferred embodiment, both the virus and the checkpoint inhibitor are administered intravenously. Preferably, the virus and the checkpoint inhibitor are administered as separate compounds. Concomitant treatment with the two agents is also possible.

In a preferred embodiment, the immune checkpoint inhibitor is administered in an amount from about 0.2 mg/kg to 50 mg/kg, more preferably about 0.2 mg/kg to 25 mg/kg.

As used herein “separate administration” or “separate” refers to a situation, wherein (a) an oncolytic adenoviral vector encoding an IL-7 as a transgene and (b) one or more immune checkpoint inhibitors are two different products or compositions distinct from each other.

Any other treatment or combination of treatments may be used in addition to the therapies of the present invention. In a specific embodiment the method or use of the invention further comprises administration of concurrent or sequential radiotherapy, chemotherapy, antiangiogenic agents or targeted therapies, such as alkylating agents, nucleoside analogs, cytoskeleton modifiers, cytostatic agents, monoclonal antibodies, kinase inhibitors or other anti-cancer drugs or interventions (including surgery) to a subject.

The terms “treat” or “increase”, as well as words stemming therefrom, as used herein, do not necessarily imply 100% or complete treatment or increase. Rather, there are varying degrees of which one of ordinary skill in the art recognizes as having a potential benefit or therapeutic effect.

It will be obvious to a person skilled in the art that, as the technology advances, the inventive concept can be implemented in various ways. The invention and its embodiments are not limited to the examples described above but may vary within the scope of the claims.

### EXPERIMENTAL SECTION

### Material and Methods

### Cell Lines

Human cancer cell lines A549 (epithelial adenocarcinoma) and RD (rhabdomyosarcoma) were purchased from the American Type Culture Collection (ATCC) (Manassas, USA). The Syrian hamster cancer cell line DDT1-MF2 (leiomyosarcoma) was a kind gift from Dr. William Wold, hamster HapT1 (pancreatic ductal adenocarcinoma cell line) was obtained from Leibniz Institute (DSMZ, Braunschweig, Germany), hamster HT100 (lung adenocarcinoma) was obtained from the Japanese Collection of Research Bioresources Cell Bank (Osaka, Japan). All cell lines were cultured under recommended conditions.

### Virus Construction

The Ad5/3-E2F-d24-hIL7 virus has been constructed by a previously described technique (Havunen et al. 2017). Tumor-specific replication was achieved by two modifications: an E2F promoter and a 24-base pair deletion in the constant region of E1A, which determines tumor selectivity regarding viral replication. Human IL7 coding sequence was introduced in E3 region replacing gp19k and 6.7k genes via bacterial artificial chromosome (BAC) recombineering strategy. The resulting viral vector sequence was confirmed by next generation sequencing.

Viral particles were produced via transfection of A549 cell line at multiplicity of infection (MOI) 1, with subsequent purification via cesium chloride gradient. The infectivity and concentration of the resulting virus were determined by TCID50 assay according to a protocol described by Lock at al. 2019.

### Cell Viability Assay

Human cell lines A549 and RD were plated in 96-well plate (flat bottom) in triplicates at 1×10⁴ cells/well for 24 hours and infected with 1, 10, 100 or 1000 VP/cell of either Ad5/3-E2F-d24 virus (also referred in the text as backbone or unarmed backbone), or Ad5/3-E2F-d24-IL7 (also referred in the text as IL7 virus, IL7 armed virus, IL7 encoding virus or IL7 oncolytic adenovirus). Similarly, hamster cell lines HT100, DDT1-MF2 and HapT1 were plated in triplicates at 1×10⁴ cells/well for 24 hours and infected with 100, 1000, 5000 or 10000 VP/cell of either backbone virus, or Ad5/3-E2F-d24-hIL7.

Cell viability was measured after 4 days (A549 and RD), 5 days (HT100 and DDT1-MF2) or 8 days (HapT1) by incubating wells for 2 hours with 20% of CellTiter 96 AQueous One Solution Proliferation Assay reagent (Promega, Wisconsin, USA). Absorbance was read at 490 nm using a Fluostar OPTIMA analyzer (BMG Labtech, Offenburg, Germany). Data was normalized to the uninfected mock control group.

### Cytokine Expression and Bioactivity Assay

The aforementioned human and hamster cell lines were infected with either 1000 VP/cell (A549 and RD) or 10000 VP/cell (HT100 and DDT1-MF2) of Ad5/3-E2F-d24-hIL7 for 3 days. Human IL7 was measured from cell supernatants using the BD Cytometric Bead Array Human Soluble Protein Master Buffer Kit (BD Biosciences, New Jersey, USA) together with human IL7 Flex Set (BD Biosciences, New Jersey, USA) according to the manufacturer's instructions. The beads were detected with the BD Accuri Flow Cytometer and the results were analyzed with FCAP Array software (version 3.0.1; BD Biosciences, New Jersey, USA).

To confirm that virally produced human IL7 was bioactive, murine IL7-dependent cell line 2E8 (ATCC, Virginia, USA) was cultured in triplicates at 2.5×10⁵ cells/ml in McCoy's 5A media (ThermoFisher, Massachusetts, USA) supplemented with 10% FBS, 1% L-glutamine and 1% Pen/strep for 24 hours. Filtered supernatant from A549 cells infected with Ad5/3-E2F-d24-hIL7 was added at 1:1, 1:4, 1:16, 1:64 and 1:256 dilutionsand were incubated for 5 days prior to viability assay using 20% of CellTiter 96 AQueous One Solution Proliferation Assay reagent as described above. Recombinant murine and human IL7 were used at 20 ng/ml concentration as positive controls.

### Animal Experiment

The efficacy of Ad5/3-E2F-d24-hIL7 was tested in vivo using immunocompetent Syrian hamsters, a model semi-permissive for adenovirus replication. Male Syrian golden hamsters (Mesocricetus auratus), 5 weeks old, (Envigo, Indiana, USA) were engrafted subcutaneously on their lower back right flank with 2×10⁶ HapT1 cells. After tumors reached 5 to 6 mm in diameter, animals were randomized and treated intratumorally with 1×10⁹ VP of either Ad5/3-E2F-d24 or Ad5/3-E2F-d24-hIL7. Control animals received intratumoral injections of PBS. Tumors were measured with digital caliper, and tumor volumes were calculated as (length×width²)/2. Hamsters received a total of 12 rounds of virus treatment before they were euthanized on day 64, and their tumors and organs were collected for subsequent analysis. Alternatively, animals were euthanized whenever the maximum allowed tumor volume (22.0 mm) was reached, tumors developed ulcers, or whenever the animals' wellbeing was compromised

### Gene Expression Assay

Fragments of animal tumor samples were preserved in RNAlater (Sigma-Aldrich, Missouri, USA), and stored at −20° C. until further use. RNA from the samples were isolated using RNeasy extraction kit (Qiagen, Hilden, Germany) according to the manufacturer's instructions and RNA concentration was measured using Qubit4 Fluorometer (ThermoFisher, Massachusetts, USA). 250 ng of purified total RNA was used to synthetize cDNA with High capacity cDNA Reverse Transcription kit (ThermoFisher, Massachusetts, USA) according to the manufacturer's instructions. Resulting cDNA was used for quantitative real-time PCR. The gene expression levels was measured using the following primers and probes: GranzymeB (forward primer 5′-CACTGTTCAGGGAGCTCAATAA-3′ (SEQ ID NO:2), reverse primer 5′-TGGAGTAGTCCTTGGGATTATAGTC-3′ (SEQ ID NO:3), probe 5′-Fam-CCTCCTTTTCTTTGATGTTGTGGGC-BBQ-3′) (SEQ ID NO:4), Perforin (forward primer 5′-TGAGTGCCCTTCTGAAATCG-3′ (SEQ ID NO:5), reverse primer 5′-TGTCGCCTGTACAGTTTTCG-3′ (SEQ ID NO:6), probe 5′-Fam-CTGGTACAGAGACCCCCACTGCAC-BBQ-3′) (SEQ ID NO:7), CD25 (forward primer 5′-TCATCAGTTTCCAGCCAGTG-3′ (SEQ ID NO:8), reverse primer 5′-GTATAAATGTCTCCATAGTTGTAGCTGC-3′ (SEQ ID NO:9), probe 5′-Fam-TCCTGAGAGTGAGACTTCCTGTCCCATC-BBQ-3′) (SEQ ID NO:10), CD137 (forward primer 5′-AGTGCATCGAGGGACTCC-3′ (SEQ ID NO:11), reverse primer 5′-CAGTTTTTACAACCCTGCTCTG-3′ (SEQ ID NO: 12), probe 5′-Fam-CTCTTGACCCGGCTTGCAATCC-BBQ-3′) (SEQ ID NO:13), interferon gamma (forward primer 5′-GAACTGGCAAAAGGCTGG-3′ (SEQ ID NO:14), reverse primer 5′-CCTTCAAGGCTTCAAAGAGTTT-3′ (SEQ ID NO:15), probe 5′-Fam-CATTGAGAGCCAGATCGTCTCCTTCTACTT-BBQ-3′) (SEQ ID NO:16), Ki67 (forward primer 5′-GACCGATCTTTTAGGTATGAAAACG-3′ (SEQ ID NO:17), reverse primer 5′-GTGGTTTTTGAAGCTTCAGCAT-3′ (SEQ ID NO:18), probe 5′-Fam-ACGGCGAGCCTCGAGAGCTAG-BBQ-3′) (SEQ ID NO:19), PD1 (forward primer 5′-GTGTCCTAGTGGGTGTCCC-3′ (SEQ ID NO:20), reverse primer 5′-GCCCTCCTTCAGAGGCT-3′ (SEQ ID NO:21), probe 5′-GCTGCTGGCCTGGGTCCTA-BBQ-3′) (SEQ ID NO:22). The results were normalized against the content of hamster gamma actin housekeeping gene cDNA and against mock (ΔΔCt). All PCR reactions were run in duplicates.

### Patient Samples Processing and Establishment of Ex Vivo Tumor Cultures

Fresh single-cell tumor digests were prepared from tumors using a protocol previously validated by our group. In brief, tumors were diced into small fragments and placed in a 50 mL falcon tube containing RPMI 1640 supplemented with 1% L-glutamine, 1% Pen/strep, collagenase type I (170 mg/L), collagenase type IV (170 mg/L), DNase I (25 mg/mL) and elastase (25 mg/mL) (all enzymes from Worthington Biochemical) for overnight enzymatic digestion with rocking at +37° C. After digestion, the cell suspension was filtered through a 70 μm filter and treated with ACK lysis buffer (Sigma-Aldrich, Missouri, USA) for the removal of undigested fragments and red blood cells. The resulting single-cell suspension was used to establish ex vivo tumor cultures by plating 3×10⁵ cells in triplicates in a 96-well plate (U-bottom) and treating them with 100 VP/cell of either Ad5/3-E2F-d24 or Ad5/3-E2F-d24-hIL7; uninfected cells were used as mock control. The cells, further used for flow cytometry, were collected on day 3 in freezing media containing 90% fetal bovine serum (FBS) and 10% dimethyl sulfoxide and stored up to −140° C. until further use.

### Cytotoxicity and Virus Replication Assay

Cell viability of ex vivo tumor cultures was determined as above described. Briefly, cell viability was measured at days 3, 5 and 7 by incubating with CellTiter 96 AQueous One Solution Proliferation Assay reagent for 2 hours. Data was normalized to uninfected control group.

In order to determine virus replication in ex vivo tumor cultures, the cells were collected in phosphate buffered saline on days 1, 2 and 3 after virus infection and the DNA was extracted with QIAmp DNA Mini Kit (Qiagen, Hilden, Germany) according to the manufacturer's instructions. Virus presence was confirmed by quantitative real-time PCR targeting the E4 region using forward primer (5′-GGAGTGCGCCGAGACAAC-3′) (SEQ ID NO:23), reverse primer (5′-ACTACGTCCGGCGTTCCAT-3′) (SEQ ID NO:24) and probe (Fam-TGGCATGACACTACGACCAACACGATCT-Tam) (SEQ ID NO:25). Quantification of E1A was calculated according to a standard curve generated using known concentrations of a plasmid coding the virus. The results were normalized to the content of human beta-actin housekeeping gene DNA. All PCR reactions were run in duplicates.

### Chemokines and Cytokines Analysis

Supernatants from the infected ex vivo tumor cultures were collected on day 3 and the presence of C-C motif ligand 2 (Ccl2), C-C motif ligand 5 (Ccl5), C-X-C motif chemokine 10 (Cxcl10), interleukin 2 (IL2), tumor necrosis factor alpha (TNFa), IFNg, interferon 1 beta (IFN1b), interleukin 4 (IL4), interleukin 6 (IL6), interleukin 10 (IL10) and transforming growth factor beta-1 (TGF-b1) was determined using the Essential Immune Response LEGENDplex panel (Biolegend, California, USA), according to the manufacturer's instructions.

The levels of C-X-C motif chemokine 9 (Cxcl9; also known as MIG) and IL7 in supernatants were determined through human MIG and IL7 flex sets (BD Biosciences, New Jersey, USA) respectively. Samples were measured in triplicates using Accuri C6 flow cytometer and analyzed through either LEGENDplex Data Analysis Software Suite (Biolegend, California, USA) or FCAP Array software. The concentration of each analyte was normalized to the total protein content measured via Qubit4 Fluorometer (ThermoFisher, Massachusetts, USA). This data was then normalized to uninfected control group.

### Flow Cytometry

Analysis of immune cell populations from ex vivo tumor cultures was performed using antibodies specific for human CD3 (clone SK7, fluorochrome Alexa Fluor 700, Biolegend, California, USA), CD4 (clone RPA-T4, fluorochrome V500, BD Biosciences, New Jersey, USA), CD8 (clone RPA-T8, fluorochrome FITC, BD Biosciences, New Jersey, USA), CD69 (clone FN50, fluorochrome PE/Cyanine7, Biolegend, California, USA), CD127 (clone HIL-7R-M21, fluorochrome PE-CF594, BD Biosciences, New Jersey, USA), GranzymeB (clone GB11, fluorochrome BV421, BD Biosciences, New Jersey, USA), Perforine (clone B-D48, fluorochrome PerCP/Cyanine5.5, Biolegend, California, USA), CD107a (clone H4A3, fluorochrome APC/Cyanine7, Biolegend, California, USA) were purchased. Intracellular staining was performed using BD Cytofix/Cytoperm Plus Kit (with BD GolgiPlug) (BD Biosciences, New Jersey, USA), according to manufacturer's instructions. All samples were stained after Fc blocking using Human TruStain FcX Receptor Blocking Solution (Biolegend, California, USA). Samples were acquired in duplicates using FACS Aria II cell sorter (BD Biosciences, New Jersey, USA) and data analysis was performed using Flowjo software v10 (Flowjo LLC, BD Biosciences, New Jersey, USA).

### Statistical Analysis

GraphPad Prism v.8.4.2 (GraphPad Software) was used for statistical analysis and graphical representation of the data. The normality of tumor progression data was performed using Shapiro-Wilk test, and the equality of variances—using Levene's test. According to these tests data was non-normal and the variances between the groups were different, so non-parametric Friedman test was used to compare multiple groups, Dunn's test was used to perform pairwise comparisons. Unpaired t-test was used to compare groups in in vitro experiments. The results were considered statistically significant when p<0.05.

### Example 1. An Oncolytic Adenovirus Armed With Human IL7 is Able to Deliver the Transgene to Cancer Cells and to Reduce the Viability of Multiple Cancer Cell Lines

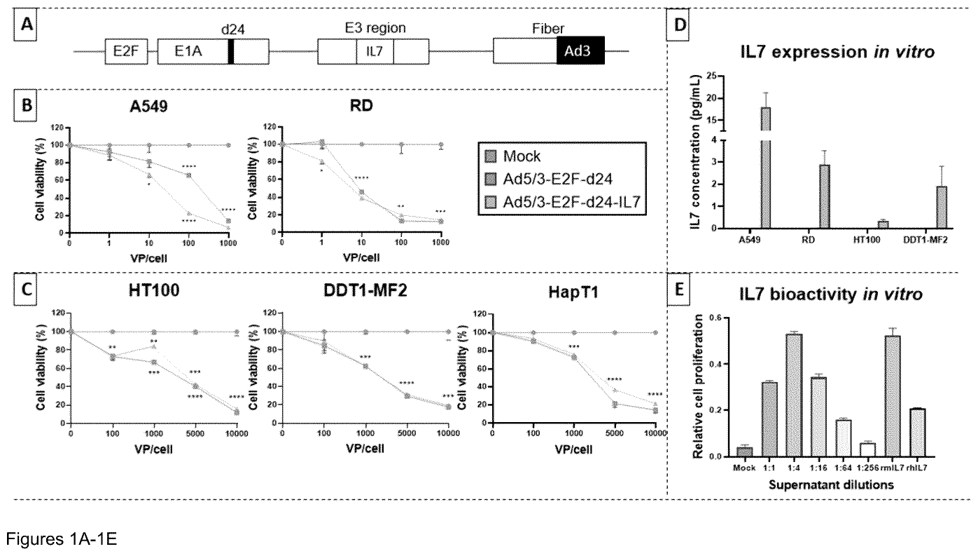

Previously we demonstrated the ability of Ad5/3-E2F-d24 viruses to penetrate and replicate in several cancer cell lines (Havunen et al. 2017). An oncolytic adenovirus coding for human IL7 utilizes the same backbone from adenovirus serotype 5 that carries the fiber knob from serotype 3, has a 24-bp deletion (d24) in the constant region 2 of the E1A gene and the insertion of a tumor-specific E2F promoter upstream the E1A region. The human IL7 gene was introduced in the partially deleted E3 gene region, making the transgene expression linked to virus replication (FIG. 1A).

The ability of Ad5/3-E2F-d24-hIL7 to infect and lyse cancer cells was evaluated in vitro using several human and hamster cell lines. Human lung and rhabdomyosarcoma cancer cell lines (A549 and RD, respectively), and hamster leiomyosarcoma, lung and pancreatic cancer cell lines (DDT1-MF2, HT100 and HapT1, respectively) were infected with different concentrations of Ad5/3-E2F-d24-hIL7; uninfected cells and cells infected with previously described (Havunen et al. 2017) unarmed Ad5/3-E2F-d24 virus were used as controls. Accordingly, the lytic efficacy of Ad5/3-E2F-d24-hIL7 virus was comparable to the unarmed virus (FIGS. 1B and 1C). These results demonstrate that the presence of the IL7 transgene does not affect virus oncolytic features.

To assess the ability of the virus to induce the transgene expression level in cancer cells, the latter were infected with Ad5/3-E2F-d24-hIL7 and supernatants were analyzed for the presence of IL7 protein levels. Interestingly, human cell lines showed higher concentration of IL7 with the maximum amount of 17.5 pg/ml produced by A549 (FIG. 1D). Hamster cell line HT100 had the lowest protein level (0.4 pg/ml), while DDT1-MF2 and RD cell lines had similar productivity (FIG. 1D).

Next, we evaluated the bioactivity of human IL7 produced by the virus. To do this, we incubated IL7-dependent murine cell line 2E8 with several dilutions of A549 supernatant with subsequent MTS cell proliferation measurement. We observed dose-dependent cell proliferation with the better outcome at 1:4 dilution compared to control group (FIG. 1E).

Overall, we confirmed that Ad5/3-E2F-d24-hIL7 is capable of killing cancer cells and inducing the expression of bioactive IL7 in the latter.

### Example 2. IL7 Oncolytic Adenovirus Promotes Tumor Regression in a Hamster Model of Pancreatic Cancer

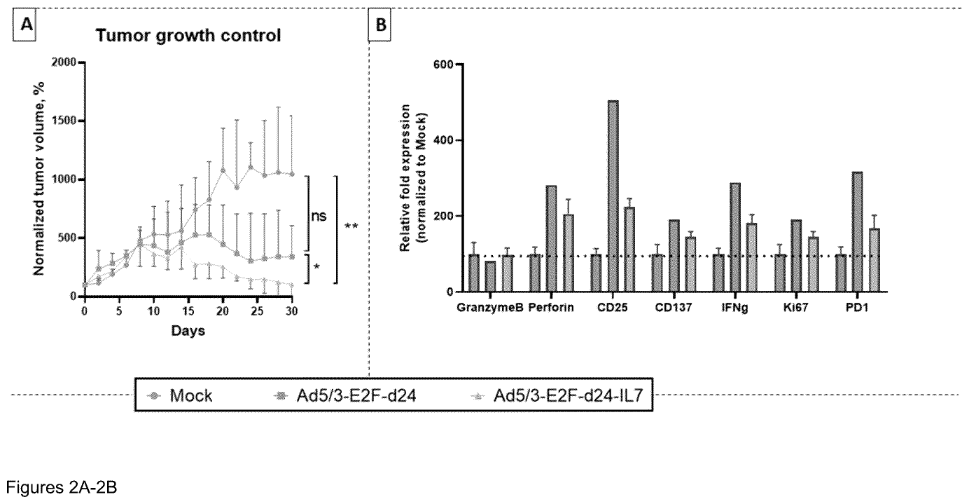

The in vivo efficacy of Ad5/3-E2F-d24-hIL7 was tested in the immunocompetent Syrian hamster, a preclinical model semi-permissive to adenovirus replication. Pancreatic cancer cell line HapT1 was engrafted subcutaneously to allow heterotopic tumor formation and subsequent intratumoral virus injections. Thirty days after treatment initiation, hamsters in the mock control group had significantly larger tumors when compared to virus-treated animals. Both backbone and IL7 coding virus-treated groups showed a trend for the reduction of tumor volumes, however IL7 coding virus-treated animals saw their tumors significantly smaller than mock and Ad5/3-E2F-d24-treated animals (p value 0.0013 and 0.0155, respectively) (FIG. 2A).

To gain a better understanding of the immunological mechanism of action of the IL7 armed adenovirus in more detail, we measured the expression level of several immune-related genes in tumors harvested from animals at day 64. We observed substantial upregulation of canonical T cell activation markers CD25, CD137, Ki67 and PD1 in both virus-treated groups, including (FIG. 2B). IFNg and Perforin expression levels were also substantially higher in virus groups potentially indicating increased cytotoxicity of immune cells in tumor microenvironment. The Ad5/3-E2F-d24-treated group showed a higher level of most analyzed cytokines. No major differences were observed in the level of GranzymeB across all groups (FIG. 2B).

In conclusion, we showed that Ad5/3-E2F-d24-hIL7 virus treatment leads to effective in vivo tumor regression and enables the activation of key immune-stimulating factors.

### Example 3. Oncolytic Adenovirus Armed With IL7 Infects and Replicates in Patient-Derived Ex Vivo Tumor Models

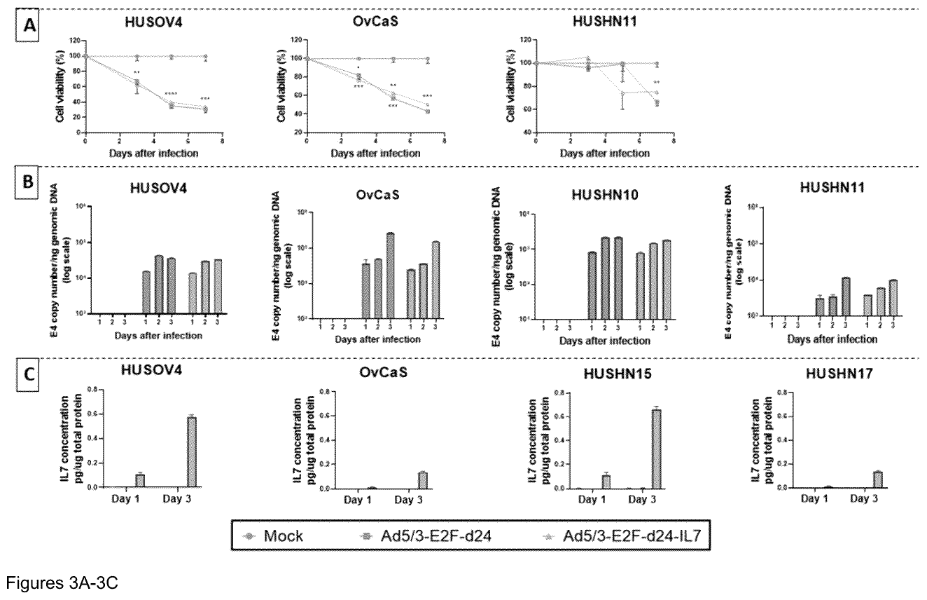

To further investigate the mechanism of action of the oncolytic adenovirus armed with IL7, we shifted towards more clinically relevant ex vivo tumor models. As expected, cell viability tests showed comparable lytic ability for both the backbone and IL-7 encoding virus in case of ovarian cancer (HUSOV4 and OvCaS): after 3 days of incubation the viability reduced to 70-80% and continued to decrease until day 7 (FIG. 3A). A reduction in viability was also observed in a head and neck sample (HUSHN11) at day 7 after virus infection with backbone and IL7 encoding virus (FIG. 3A).

Next, we evaluated the virus copy number in ex vivo tumor samples via quantitative real-time PCR targeting the E4 gene of the adenovirus. Overall, the amount of the viruses was comparable between the backbone and IL-7 encoding virus across all tested samples, with an overall trend towards increased of viral genome copies over time (FIG. 3B). However, there was clear difference in the total viral copy number between sample cancer types. OvCaS and HUSHN10 samples had highest amount of virus DNA, while in HUSHN11 it was 100 times lower. HUSOV4 showed moderate virus copy number (FIG. 3B).

Finally, we measured the human IL7 concentration in supernatants. In all tested samples, we saw an increase in IL7 concentration from day 1 to day 3, however, as expected, the amount of protein was different across the samples. HUSOV4 and HHUSHN15 samples had higher amount of IL7 reaching 0.65 and 0.57 pg/ug of total IL7 protein respectively, while IL7 concentration in OvCaS and HUSHN17 samples was 3 times lower (FIG. 3C).

Overall, this data shows the ability of IL7 oncolytic adenovirus to infect patient-derived tumor digests, replicate and lyse cancer cells, despite the differences between the samples.

### Example 4. Oncolytic Adenovirus Armed With IL7 Converts Local Tumor Microenvironment Towards Immunostimulation

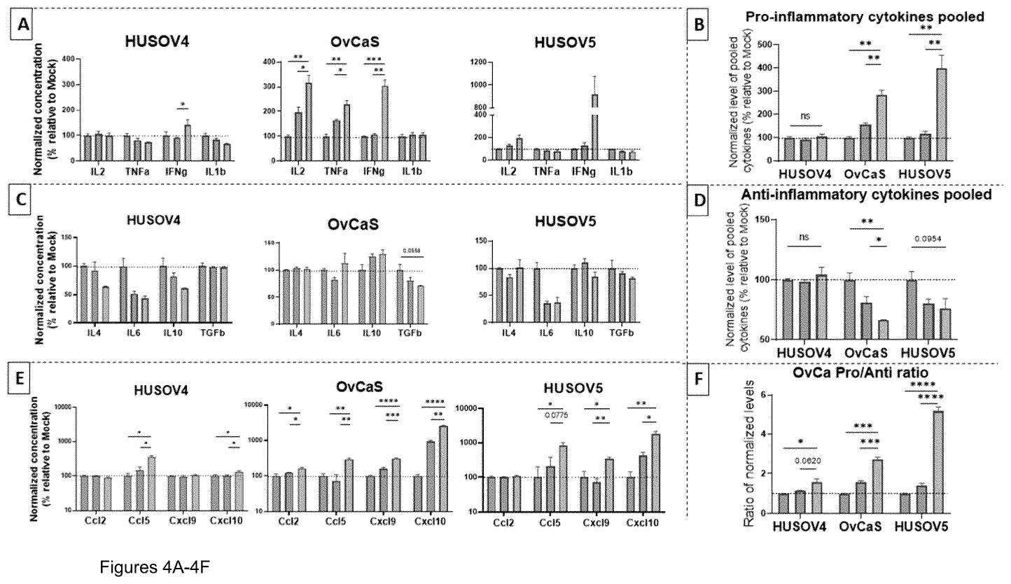

In order to evaluate the immune status of infected patient-derived ex vivo ovarian cancer samples, we measured the concentration of key immune signaling molecules—chemokines and cytokines. First, we compared the level of pro-inflammatory cytokines: IL2, TNFa, IFNg and IL1b. All tested samples showed significant increase of IFNg when infected with IL7 encoding virus group compared to uninfected cells and backbone virus group. When infected with IL7 encoding virus, OvCaS and HUSOV5 samples also displayed higher expression of IL2, while HUSOV4 did not exhibit marked cytokine changes across the different experimental groups. Moreover, OvCaS sample saw remarkably higher concentration of TNFa in IL7 encoding virus group than any other treatment group. Interestingly, no changes in the level of IL1b was observed in all tested samples (FIG. 4A). Overall, the level of pooled pro-inflammatory cytokines in OvCaS and HUSOV5 was notably higher in IL7 virus-treated group compared to other groups (FIG. 4B).

Next, we measured the level of anti-inflammatory cytokines: IL4, IL6, IL10 and TGFb. Overall, no significant increase of cytokine levels was observed in virus-treated groups (FIG. 4C) compared to uninfected cells. HUSOV4 demonstrated a notable reduction in IL4, IL6 and IL10 concentration, while OvCaS and HUSOV5 had reduced amount of TGFb, upon infection with IL7 encoding virus. The level of pooled anti-inflammatory cytokines was decreased in 2 out of 3 samples (OvCaS and HUSOV5) in cultures infected with the IL7 encoding virus. The reduction was statistically significant between backbone virus and IL7 encoding virus in the OvCaS sample (FIG. 4D).

Lastly, we measured the level of chemokines in ex vivo infected samples: Ccl2, Ccl5, Cxcl9, Cxcl10. Most notably, virus-treated samples showed remarkable increase in Ccl5 and Cxcl10 production, wherein IL7 encoding virus yielded significantly higher production than the backbone (FIG. 4E). HUSOV4 sample did not show any other changes in chemokines concentration across the groups, while OvCaS sample showed upregulation of Ccl2 and Cxcl9 when treated with IL7 encoding virus. HUSOV5 sample showed notable upregulation of Ccl5, Cxcl9 and Cxcl10 upon infection with IL7 encoding virus.

Importantly, the IL-7 encoding virus induced the highest prevalence of pro-inflammatory cytokines over anti-inflammatory ones in most tested ovarian cancer patient samples (FIG. 4F). Overall, these data showed the ability of Ad5/3-E2F-d24-hIL7 to induce the production of signaling molecules for converting the tumor microenvironment towards proinflammation, which is particularly useful for the recruitment of T-cells to the tumors.

### Example 5. Oncolytic Adenovirus Armed With IL7 Activates Infiltrating CD4+ and CD8+ T Cells

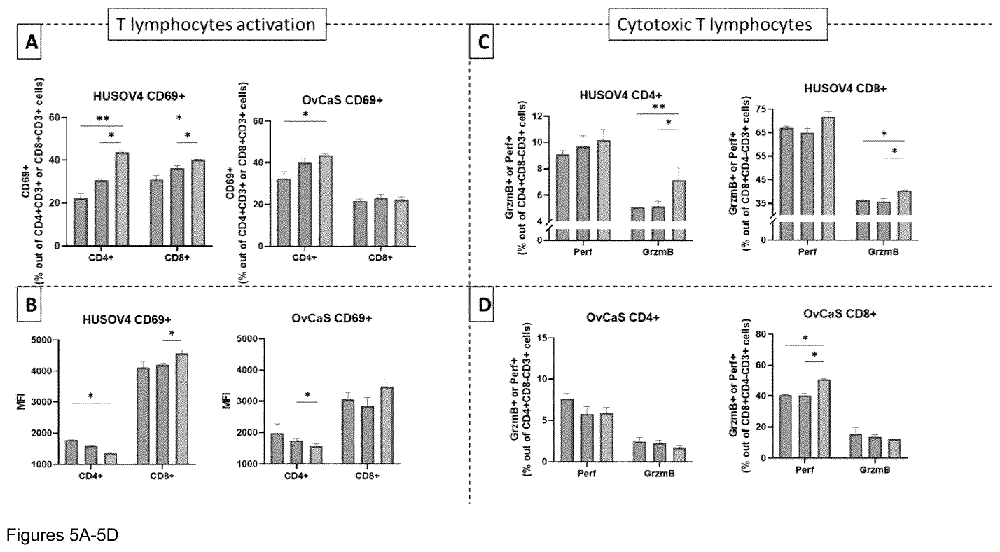

To evaluate the effect of Ad5/3-E2F-d24-hIL7 on tumor-infiltrating lymphocytes, we first verified the expression level of activation and cytotoxic markers separately on CD4+ and CD8+ cells from HUSOV4 and OvCaS ex vivo tumor cultures treated with the oncolytic adenoviruses via flow cytometry. Upon infection with the IL7 encoding virus, HUSOV4 cultures showed a significant increase in the amount of CD69+CD4+ T cells and CD69+CD8+ T cells compared to other groups (FIG. 5A), while CD8+ cells also had higher expression of CD69+ receptors on their surface (FIG. 5B). Similar scenario was observed in IL7 encoding virus-infected OvCaS cultures with an observed increase in CD69+CD4+ T cells compared to uninfected cultures, however, no significant changes were detected in CD8+ T cells (FIG. 5A).

Moreover, we evaluated the changes in the number of cytotoxic T cells expressing Perforin (Perf) and GranzymeB (GrzmB), two potent cytolytic agents. In HUSOV4 cultures treated with IL7 encoding virus, we observed a notable increase of GrzmB+ T cells, both CD4+ and CD8+ (FIG. 5C), while no changes in GrzmB+ cells in OvCaS sample was detected (FIG. 5D). However, in the same sample we detected the increase in the CD8+Perf+ cells in IL7 group (FIG. 5D), and observed an increased frequency of both CD4+Perf+ and CD8+Perf+ populations in HUSOV4 treated with IL7 encoding virus.

Overall, our data shows that Ad5/3-E2F-d24-hIL7 is able to activate infiltrating lymphocytes and increase the proportion of cytotoxic CD4+ and CD8+ cells.

### Example 6. Oncolytic Adenovirus Armed With IL7 in Combination With Human Immune Checkpoint Inhibitors (PD-1 and/or PDL-1)

### Virus Construct and Immune Checkpoint Inhibitors (ICIs)

The virus used in this proof-of-concept study is TILT-517 (Ad5/3-E2F-d24-hIL7). Virus was prepared by bacterial artificial chromosomal recombineering techniques, incorporating modifications on E2F promoter and a 24 base-pair deletion in the constant region of E1A as well as human interleukin-7 protein as a transgene. ICIs, such as human anti-PD-L1 Tecentriq® (Atezolizumab, Roche; referred as antiPD-L1 in the text) and human anti-PD-1 KEYTRUDA® (Pembrolizumab, Merk; referred as antiPD-1 in the text), were used in the study either alone and/or in combination with TILT-517.

### Patient Sample Processing and Ex-Vivo Tumor Cultures

A tumor sample from a patient with renal cell carcinoma (RCC) was obtained from Helsinki University Hospital (HUS). In brief, tumors were diced into small fragments and placed in a 50 mL falcon tube containing RPMI 1640 supplemented with 1% L-glutamine, 1% Pen/strep, collagenase type I (170 mg/L), collagenase type IV (170 mg/L), DNase I (25 mg/mL) and elastase (25 mg/mL) (all enzymes from Worthington Biochemical) for overnight enzymatic digestion with rocking at +37° C. After digestion, the cell suspension was filtered through a 70 μm filter and treated with ACK lysis buffer (Sigma-Aldrich, Missouri, USA) for the removal of undigested fragments and red blood cells. All this process resulted in a heterogeneous single cell suspension that later was used for establishing ex-vivo tumor cultures to use in cell viability assays, in this case, HUSRenca5.

### Cell Viability Assays

The MTS assay was used to access the cell viability of infected tumor cells. 20,000 viable patient tumor cells were plated in triplicates in a 96 well plate (U-bottom). Next day, tumors were treated either with TILT-517, antiPD-1, antiPDL-1, TILT-517 + antiPD-1 or TILT-517 + antiPD-L1. Uninfected cells were used as a Mock control. Virus was infected in a concentration of 100 VP/cell. The concentration of both ICls used was 0.1 mg/ml. Cell viability was assessed after days 1, 2, 3, 4, and 5 post treatment, by the incubation with CellTiter 96 AQueous One Solution Proliferation Assay reagent (Promega, Wisconsin, USA) for 2 hours. Absorbance was read at 490 nm using Hidex Sense plate reader (Hidex, Turku, Finland). Data was normalized to the uninfected mock control group.

### Statistical Analysis

GraphPad Prism v9.2.0 (GraphPad Software) was used for statistical analysis and graphical representation of the data. Statistical test was run by using unpaired t-test between groups, and significance was considered when p<0.05.

### Results

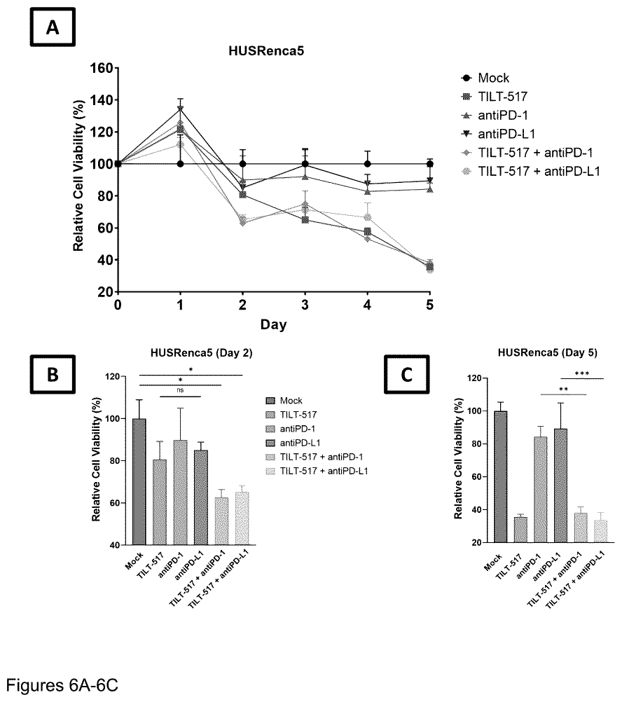

The results in FIG. 6A show that the use of TILT-517 and its combination with ICls works more effectively compared to the use of either antiPD-1 or antiPD-L1 alone in an RCC sample. It is important to remark that ICls are a common immunotherapy used as a standard of care for RCC. Yet, as it can be observed in FIG. 6A, antiPD-1 and antiPD-L1 treatment decreases the relative cell viability of the tumor culture to only around 90%, whereas TILT-517 involved groups continue to decrease the relative cell viability of the tumor as days go by, even reaching below 40%.

Of note, at Day 2, tumor killing mediated by the combination group of TILT-517+ antiPD-1 and TILT-517+ antiPD-L1 is statistically significant compared to Mock (FIG. 6B). Importantly, the combination groups showed to be a promising strategy as they display more than 20% of improvement in decreasing the sample cell viability of the cultures compared to TILT-517, antiPD-1 or antiPD-L1 monotherapies (FIG. 6B). By day 5, the combination groups showed statistically significant higher tumor cell killing compared to either antiPD-1 or antiPD-L1 alone (FIG. 6C). One possible mechanism behind the better tumor killing by the combination treatment group is due to higher immune cell activation.

Overall, the quick and effective response of TILT-517 + antiPD-L1 or antiPD-1 compared to either therapies alone suggest a promising approach for clinical implementation.

### REFERENCES

### Cited Non-Patent Publications

Havunen R, Siurala M, Sorsa S, Gronberg-Vaha-Koskela S, Behr M, Tahtinen S, Santos JM, Karell P, Rusanen J, Nettelbeck DM, et al.: Oncolytic Adenoviruses Armed with Tumor Necrosis Factor Alpha and Interleukin-2 Enable Successful Adoptive Cell Therapy. Mol Ther Oncolytics 2017, 4:77-86.
Heninger AK, Theil A, Wilhelm C, Petzold C, Huebel N, Kretschmer K, Bonifacio E, Monti P: IL-7 abrogates suppressive activity of human CD4+CD25+FOXP3+ regulatory T cells and allows expansion of alloreactive and autoreactive T cells. J Immunol 2012, 189:5649-5658.
Huang J, Zheng M, Zhang Z, Tang X, Chen Y, Peng A, Peng X, Tong A, Zhou L: Interleukin-7-loaded oncolytic adenovirus improves CAR-T cell therapy for glioblastoma. Cancer Immunology, Immunotherapy 2021, 70:2453-2465.
Lock, M., et al., Measuring the Infectious Titer of Recombinant Adenovirus Using Tissue Culture Infection Dose 50% (TCID (50)) End-Point Dilution and Quantitative Polymerase Chain Reaction (qPCR). Cold Spring Harb Protoc, 2019. 2019 (8).
Nakao S, Arai Y, Tasaki M, Yamashita M, Murakami R, Kawase T, Amino N, Nakatake M, Kurosaki H, Mori M, et al.: Intratumoral expression of IL-7 and IL-12 using an oncolytic virus increases systemic sensitivity to immune checkpoint blockade. Science Translational Medicine 2020, 12:eaax7992.
Pellegrini M, Calzascia T, Elford AR, Shahinian A, Lin AE, Dissanayake D, Dhanji S, Nguyen LT, Gronski MA, Morre M, et al.: Adjuvant IL-7 antagonizes multiple cellular and molecular inhibitory networks to enhance immunotherapies. Nat Med 2009, 15:528-536.
Rosenberg SA, Sportès C, Ahmadzadeh M, Fry TJ, Ngo LT, Schwarz SL, Stetler-Stevenson M, Morton KE, Mavroukakis SA, Morre M, et al.: IL-7 administration to humans leads to expansion of CD8+ and CD4+ cells but a relative decrease of CD4+ T-regulatory cells. Journal of immunotherapy (Hagerstown, Md.: 1997) 2006, 29:313-319.
Sportès C, Babb RR, Krumlauf MC, Hakim FT, Steinberg SM, Chow CK, Brown MR, Fleisher TA, Noel P, Maric I, et al.: Phase I study of recombinant human interleukin-7 administration in subjects with refractory malignancy. Clin Cancer Res 2010, 16:727-735.
Vudattu, N., Magalhaes, I., Hoehn, H., Pan D., and Maeurer M.J. (2009). Expression analysis and functional activity of interleukin-7 splice variants. Genes Immun 10, 132-140.
White, E., Sabbatini, P., Debbas, M., Wold, W.S.M., Kusher, D.I., and Gooding, L. (1992). The 19-kilodalton adenovirus E1B transforming protein inhibits programmed cell death and prevents cytolysis by tumor necrosis factor alpha. Mol. Cell. Biol. 1992; 12:2570-2580.

### Cited Patent Publications

WO2014170389
WO2016146894
WO2020249873
EP 3858369

1. An oncolytic adenoviral vector comprising a nucleic acid sequence encoding an interleukin 7 (IL-7) polypeptide or a variant thereof as a transgene, wherein a backbone of the oncolytic adenoviral vector is an adenovirus serotype 5 (Ad5) backbone with the fiber knob of adenovirus serotype 3 (Ad3).

2. The oncolytic adenoviral vector according to claim 1, wherein said nucleic acid sequence encoding an interleukin 7 (IL-7) polypeptide or a variant thereof is in the place of a deleted nucleic acid sequence in the E3 region of said oncolytic adenoviral vector.

3. The oncolytic adenoviral vector according to claim 2, wherein the deletion of a nucleic acid sequence in the E3 region is a deletion of viral gp19k and 6.7k reading frames.

4. The oncolytic adenoviral vector according to claim 1, wherein the vector comprises a 24 bp deletion (Δ24) in the adenoviral E1 sequence of said oncolytic adenoviral vector.

5. The oncolytic adenoviral vector according to claim 1, wherein the vector comprises Ad5/3-E2F-d24 backbone.

6. The oncolytic adenoviral vector according to claim 5, wherein the vector has the structure Ad5/3-E2F-d24-IL-7.

7. The oncolytic adenoviral vector according to claim 1, wherein the vector comprises nucleic acid sequence encoding a further transgene.

8. The oncolytic adenoviral vector according to claim 7, wherein the further transgene is encoding a cytokine.

9. The oncolytic adenoviral vector according to claim 8, wherein the cytokine is selected from the group consisting of: TNFalpha, interferon alpha, interferon beta, interferon gamma, complement C5a, CD40L, IL-12, IL-23, IL-21, IL-15, IL-17, IL-18, IL-2, CCL1, CCL11, CCL12, CCL13, CCL14-1, CCL14-2, CCL14-3, CCL15-1, CCL15-2, CCL16, CCL17, CCL18, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5 (=RANTES), CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCR1, CXCR2, CXCR4, CXCR5, CXCR6, CXCR7 and XCL2.

10. The oncolytic adenoviral vector according to claim 9, wherein the cytokine is TNFalpha or IL-15.

11. A pharmaceutical composition comprising an oncolytic adenoviral vector according to claim 1 and at least one of the following: physiologically acceptable carriers, buffers, excipients, adjuvants, additives, antiseptics, preservatives, filling, stabilising and/or thickening agents.

12. A method for treating a cancer or tumor in a subject comprising administering a composition comprising the oncolytic vector of claim 1 to the subject.

13. The method according to claim 12, wherein the cancer or tumor is selected from a group consisting of nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheo-chromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, esophagus cancer, gall bladder cancer, head cancer, eye cancer, neck cancer, kidney cancer, Wilms' tumor, Kaposi's sarcoma, prostate cancer, testicular cancer, Hodgkin's disease, non-Hodgkin's lymphoma, skin cancer, mesothelioma, multiple myeloma, ovarian cancer, endocrine pancreatic cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer, and tonsil cancer.

14. The method according to claim 12 or 13 together with an adoptive cell therapeutic composition.

15. The method according to claim 12, wherein the method comprises administering the composition comprising the oncolytic adenoviral vector together with an immune checkpoint inhibitor to the subject.

16. The method according to claim 15, wherein said immune checkpoint inhibitor selectively binds to PD-L1 or PD1.

17. The method according to claim 16, wherein the immune checkpoint inhibitor selectively binding to PD-L1 or PD-1 is selected from the group consisting of: BMS-936559, LY3300054, atezolizumab, durvalumab, avelumab, envafolimab, cosibelimab, pembrolizumab, nivolumab, cemiplimab, sintilimab, tislelizumab, spartalizumab, toripalimab, dostarlimab, INCMGA00012 and AMP-514.

18. The method according to claim 12, wherein the method comprises administering the composition comprising the oncolytic adenoviral vector together with radiotherapy, monoclonal antibodies, chemotherapy, small molecular inhibitors, hormonal therapy or other anti-cancer drugs or interventions to the subject.

19. The oncolytic method according to claim 12, wherein the method comprises administering the composition comprising the oncolytic adenoviral vector together with an immune checkpoint inhibitor, wherein said oncolytic adenoviral vector has the structure Ad5/3-E2F-d24-IL-7, and wherein said immune checkpoint inhibitor selectively binds to PD-L1 or PD-1.
